SEQUENCE LISTING (71 sequences)

SEQ ID NO: 1 - protein, 125 aa, synthetic construct
  REGION 1..125: note = 73-2 VH amino acid sequence - Artificial sequence translated froman isolated clone of an RNA display library
EVQLVESGGGLVQPGGSLRLSCAASGFTFSSYYMHWVRQAPGKGLEWVSYISGSGSSTYYADSVKGRFTISRDNSKNTLYLQMNSLRAEDTAVYYCARDRMLVDSVNRVFGFDVWGQGTLVTVSS

SEQ ID NO: 2 - protein, 107 aa, synthetic construct
  REGION 1..107: note = 73-2 VL amino acid sequence - Artificial sequence translated froman isolated clone of an RNA display library
DIQMTQSPSSLSASVGDRVTITCQASQDISNYLNWYQQKPGKAPKLLIYDASNLETGVPSRFSGSGSGTDFTFTISSLQPEDIATYYCQQHTNFPFTFGQGTKVEIK

SEQ ID NO: 3 - protein, 125 aa, synthetic construct
  REGION 1..125: note = 74-1 VH amino acid sequence - Artificial sequence translated froman isolated clone of an RNA display library
EVQLVESGGGLVQPGGSLRLSCAASGFTVSSYYMHWVRQAPGKGLEWVSYISGSGSSTYYADSVKGRFTISRDNSKNTLYLQMNSLRAEDTAVYYCARDRMLVDSVNRVFGFDVWGQGTLVTVSS

SEQ ID NO: 4 - protein, 107 aa, synthetic construct; identical to SEQ ID NO: 2
  REGION 1..107: note = 74-1 VL amino acid sequence - Artificial sequence translated froman isolated clone of an RNA display library

SEQ ID NO: 5 - protein, 124 aa, synthetic construct
  REGION 1..124: note = 78-1 VH amino acid sequence - Artificial sequence translated froman isolated clone of an RNA display library
EVQLVESGGGLVQPGGSLRLSCAASGFTFSSYYMHWVRQAPGKGLEWVSAISTSGRSTYYADSVKGRFTISRDNSKNTLYLQMNSLRAEDTAVYYCARGRRAGMVAFWDGFDVWGQGTLVTVSS

SEQ ID NO: 6 - protein, 107 aa, synthetic construct
  REGION 1..107: note = 78-1 VL amino acid sequence - Artificial sequence translated froman isolated clone of an RNA display library
DIQMTQSPSSLSASVGDRVTITCQASQDISNYLNWYQQKPGKAPKLLIYDASNLETGVPSRFSGSGSGTDFTFTISSLQPEDIATYYCQQYDTLPFTFGQGTKVEIK

SEQ ID NO: 7 - protein, 125 aa, synthetic construct
  REGION 1..125: note = 78-2 VH amino acid sequence - Artificial sequence translated froman isolated clone of an RNA display library
MEVQLVESGGGLVQPGGSLRLSCAASGFTFASYAMHWVRQAPGKGLEWVSIISGSGGSTDYADSVKGRFTISRDNSKNTLYLQMNSLRAEDTAVYYCARGRHIPVSRFNAAFDVWGQGTLVTVSS

SEQ ID NO: 8 - protein, 107 aa, synthetic construct
  REGION 1..107: note = 78-2 VL amino acid sequence - Artificial sequence translated froman isolated clone of an RNA display library
DIQMTQSPSSLSASVGDRVTITCQASQDISNYLNWYQQKPGKAPKLLIYDASNLETGVPSRFSGSGSGTDFTFTISSLQPEDIATYYCQQYASWPWTFGQGTKVEIK

SEQ ID NO: 9 - protein, 123 aa, synthetic construct
  REGION 1..123: note = 78-5 VH amino acid sequence - Artificial sequence translated froman isolated clone of an RNA display library
EVQLVESGGGLVQPGGSLRLSCAASGFTFSSYAMNWVRQAPGKGLEWVSAIWGSGGYTYYADSVKGRFTISRDNSKNTLYLQMNSLRAEDTAVYYCARDRVESFRTNKGFDVWGQGTLVTVSS

SEQ ID NO: 10 - protein, 107 aa, synthetic construct
  REGION 1..107: note = 78-5 VL amino acid sequence - Artificial sequence
DIQMTQSPSSLSASVGDRVTITCQASQDISNYLNWYQQKPGKAPKLLIYDASNLETGVPSRFSGSGSGTDFTFTISSLQPEDIATYYCQQFNNHPFTFGQGTKVEIK

SEQ ID NO: 11 - protein, 124 aa, synthetic construct
  REGION 1..124: note = 78-8 VH amino acid sequence - Artificial sequence translated froman isolated clone of an RNA display library
EVQLVESGGGLVQPGGSLRLSCAASGFTFSSYVMHWVRQAPGKGLEWVSAISGSGGGTYYADSVKGRFTISRDNSKNTLYLQMNSLRAEDTAVYYCARGRLIGLALYGEAFDVWGQGTLVTVSS

SEQ ID NO: 12 - protein, 107 aa, synthetic construct
  REGION 1..107: note = 78-8 VL amino acid sequence - Artificial sequence translated froman isolated clone of an RNA display library
DIQMTQSPSSLSASVGDRVTITCQASKDISNYLNWYQQKPGKAPKLLIYDASNLETGVPSRFSGSGSGTDFTFTISSLQPEDIATYYCQQFNSFPLTFGQGTKVEIK

SEQ ID NO: 13 - protein, 124 aa, synthetic construct
  REGION 1..124: note = 78-8D2 VH amino acid sequence - Artificial sequence translatedfrom an isolated clone of an RNA display library
EVQLVESGGGLVQPGGSLRLSCAASGFTFSSYVMHWVRQAPGKGLEWVSAITWSGRGTYYADSVKGRFTISRDNSKNTLYLQMNSLRAEDTAVYYCARGRLFGLALYGEAFDVWGQGTLVTVSS

SEQ ID NO: 14 - protein, 107 aa, synthetic construct
  REGION 1..107: note = 78-8D2 VL amino acid sequence - Artificial sequence translatedfrom an isolated clone of an RNA display library
DIQMTQSPSSLSASVGDRVTITCQASKDISNYLNWYQQKPGKAPKLLIYDASNLETGVPSRFSGSGSGTDFTFIISSLQPEDIATYYCQQFNSFPLTFGQGTKVEIK

SEQ ID NO: 15 - protein, 124 aa, synthetic construct
  REGION 1..124: note = 78-8D4 VH amino acid sequence - Artificial sequence translatedfrom an isolated clone of an RNA display library
EVQLVESGGGLVQPGGSLRLSCAASGFTFSSYVMHWVRQAPGKGLEWVSAITWSGGGTYYADSVKGRFTISRDNSKNTLYLQMNSLRAEDTAVYYCARGRLFGLVRYGEAFDVWGQGTLVTVSS

SEQ ID NO: 16 - protein, 107 aa, synthetic construct; identical to SEQ ID NO: 12
  REGION 1..107: note = 78-8D4 VL amino acid sequence - Artificial sequence translatedfrom an isolated clone of an RNA display library

SEQ ID NO: 17 - protein, 124 aa, synthetic construct
  REGION 1..124: note = 78-8D5 VH amino acid sequence - Artificial sequence translatedfrom an isolated clone of an RNA display library
EVQLVESGGGLVQPGGSLRLSCAASGFTFSSYVMHWVRQAPGKGLEWVSAISWSGGGTYYADSVKGRFTISRDNSKNTLYLQMNSLRAEDTAVYYCARGRLFGLALYGEAFDVWGQGTLVTVSS

SEQ ID NO: 18 - protein, 107 aa, synthetic construct; identical to SEQ ID NO: 12
  REGION 1..107: note = 78-8D5 VL amino acid sequence - Artificial sequence translatedfrom an isolated clone of an RNA display library

SEQ ID NO: 19 - protein, 124 aa, synthetic construct
  REGION 1..124: note = 78-8D6 VH amino acid sequence - Artificial sequence translatedfrom an isolated clone of an RNA display library
EVQLVESGGGLVQPGGSLRLSCVASGFTFSSYVMHWVRQAPGKGLEWVSAISWSGGGTYYADSVKGRFTISRDNSKNTLYLQMNSLRAEDTAVYYCARGRLFGLALYGEAFDVWGQGTLVTVSS

SEQ ID NO: 20 - protein, 107 aa, synthetic construct
  REGION 1..107: note = 78-8D6 VL amino acid sequence - Artificial sequence translatedfrom an isolated clone of an RNA display library
DIQMTQSPSSLSASVGDRVTITCQASKDISNYLNWYQQKPGKAPKLLIYNASNLETGVPSRFSGSGSGTDFTFTISSLQPEDIATYYCQQFNSFPLTFGQGTKVEIK

SEQ ID NO: 21 - protein, 124 aa, synthetic construct
  REGION 1..124: note = 78-8D7 VH amino acid sequence - Artificial sequence translatedfrom an isolated clone of an RNA display library
EVQLVESGGGLVQPGGSLRLSCAASGFTFSSYVMHWVRQAPGKGLEWVSAISWSGAGTYYADSVKGRFTISRDNSKNTLYLQMNSLRAEDTAVYYCARGRLFGRALYGEAFDVWGQGTLVTVSS

SEQ ID NO: 22 - protein, 107 aa, synthetic construct
  REGION 1..107: note = 78-8D7 VL amino acid sequence - Artificial sequence translatedfrom an isolated clone of an RNA display library
DIQMTQSPSSLSASVGDRVTITCQASKDISIYLNWYQQKPGKAPKLLIYDAFNLETGVSSRFSGSGSGTDFTFTISSLQPEDIATYYCQQFNSFPLTFGQGTKVEIK

SEQ ID NO: 23 - protein, 125 aa, synthetic construct
  REGION 1..125: note = 78-8D10 VH amino acid sequence - Artificial sequence translatedfrom an isolated clone of an RNA display library
MEVQLVESGGGLVQPGGSLRLSCAASGFTFSSYVMHWVRQAPGKGLEWVSAISWSGRGTYYADSVKGRFTISRDNSKNTLYLQMNSLRAEDTAVYYCARGRLFGLALYGEAFDVWGQGTLVTVSS

SEQ ID NO: 24 - protein, 107 aa, synthetic construct; identical to SEQ ID NO: 12
  REGION 1..107: note = 78-8D10 VL amino acid sequence - Artificial sequence translatedfrom an isolated clone of an RNA display library

SEQ ID NO: 25 - protein, 124 aa, synthetic construct
  REGION 1..124: note = 78-8D14 VH amino acid sequence - Artificial sequence translatedfrom an isolated clone of an RNA display library
EVQLVESGGGLVQPGGSLRLSCAASGFTFSSYVMHWVRQAPGKGLEWVSAISWSGRGTYYADSVKGRFTISRDNSKNTLYLQMNSLRAEDTAVYYCARGRLFGLARYGEAFDVWGQGTLVTVSS

SEQ ID NO: 26 - protein, 107 aa, synthetic construct
  REGION 1..107: note = 78-8D14 VL amino acid sequence - Artificial sequence translatedfrom an isolated clone of an RNA display library
DIQMTQSPSSLSASVGDRVTITCQASRDISNYLNWYQQKPGKAPKLLIYDASNLETGVPSRFSGSGSGTDFTFTISSLQPEDIATYYCQQFNSFPLTFGQGTKVEIK

SEQ ID NO: 27 - protein, 124 aa, synthetic construct
  REGION 1..124: note = 78-8D22 VH amino acid sequence - Artificial sequence translatedfrom an isolated clone of an RNA display library
EVQLVESGGGLVQPGGSLRLSCAASGFTFSSYVMHWVRQAPGKGLEWVSAISWSGSGTYYADSVKGRFTISRDNSKNTLYLQMNSLRAEDTAVYYCARGRLFGLARYGEAFDVWGQGTLVTVSS

SEQ ID NO: 28 - protein, 107 aa, synthetic construct
  REGION 1..107: note = 78-8D22 VL amino acid sequence - Artificial sequence translatedfrom an isolated clone of an RNA display library
DIQMTQSPSSLSASVGDRVTITCQASKDISNYLNWYQQKPGKAPKLLIYDASNLETGVPSRFSGSGSGTDFTFTISSLQPEDIATYYCQQFNSFALTFGQGTKVEIK

SEQ ID NO: 29 - protein, 120 aa, synthetic construct
  REGION 1..120: note = 78-13 VH amino acid sequence - Artificial sequence translatedfrom an isolated clone of an RNA display library
EVQLVESGGGLVQPGGSLRLSCAASGFTFSSYAMHWVRQAPGKGLEWVSIISGSGRSTYYADSVKGRFTISRDNSKNTLYLQMNSLRAEDTAVYYCARDRNRNTYGFDVWGQGTLVTVSS

SEQ ID NO: 30 - protein, 107 aa, synthetic construct
  REGION 1..107: note = 78-13 VL amino acid sequence - Artificial sequence translatedfrom an isolated clone of an RNA display library
DIQMTQSPSSLSASVGDRVTITCQASQDISNYLNWYQQKPGKAPKLLIYDASNLETGVPSRFSGSGSGTDFTFTISSLQPEDIATYYCQQRSSLPYTFGQGTKVEIK

SEQ ID NO: 31 - protein, 125 aa, synthetic construct
  REGION 1..125: note = 78-19 VH amino acid sequence - Artificial sequence translatedfrom an isolated clone of an RNA display library
EVQLVESGGGLVQPGGSLRLSCAASGFTFSSYVMHWVRQAPGKGLEWVSAINGNGASTYYADSVKGRFTISRDNSKNTLYLQMNSLRAEDTAVYYCARDRYYTHRYYPQYGFDVWGQGTLVTVSS

SEQ ID NO: 32 - protein, 107 aa, synthetic construct
  REGION 1..107: note = 78-19 VL amino acid sequence - Artificial sequence translatedfrom an isolated clone of an RNA display library; X is any aminoacid
  REGION 90..91: note = misc_feature - Xaa can be any naturally occurring amino acid
  SITE 93: note = misc_feature - Xaa can be any naturally occurring amino acid
DIQMTQSPSSLSASVGDRVTITCQASQDISNYLNWYQQKPGKAPKLLIYDASNLETGVPSRFSGSGSGTDFTFTISSLQPEDIATYYCQXXYXVPYTFGQGTKVEIK

SEQ ID NO: 33 - protein, 125 aa, synthetic construct
  REGION 1..125: note = 78-24 VH amino acid sequence - Artificial sequence translatedfrom an isolated clone of an RNA display library
MEVQLVESGGGLVQPGGSLRLSCAASGFTFSSYSMHWVRQAPGKGLEWVSGISGSGGSTSYAYSVKGRFTISRDNSKNTLYLQMNSLRAEDTAVYYCARDLMSFNLTRTGGFDVWGQGTLVTVSS

SEQ ID NO: 34 - protein, 107 aa, synthetic construct
  REGION 1..107: note = 78-24 VL amino acid sequence - Artificial sequence translatedfrom an isolated clone of an RNA display library
DIQMTQSPSSLSASVGDRVTITCQASQDISNYLNWYQQKPGKAPKLLIYDASNLETGVPSRFSGSGSGTDFTFTISSLQPEDIATYYCQQGDAWPWTFGQGTKVEIK

SEQ ID NO: 35 - protein, 124 aa, synthetic construct
  REGION 1..124: note = 78-24D1 VH amino acid sequence - Artificial sequence translatedfrom an isolated clone of an RNA display library
EVQLVESGGGLVQPGGSLRLSCAASGFTFTSYSMHWVRQAPGKGLEWVSGISGSGRSTSYADSVKGRFTISRDNSKNTLYLQMNSLRAEDTAVYYCARDLMAFNLTRAGGFDVWGQGTLVTVSS

SEQ ID NO: 36 - protein, 124 aa, synthetic construct
  REGION 1..124: note = 78-24D1V VH amino acid sequence - Artificial sequence translatedfrom an isolated clone of an RNA display library
EVQLVESGGGLVQPGGSLRLSCAASGFTFTSYSMHWVRQAPGKGLEWVSGISGSGRSTSYADSVKGRFTISRDNSKNTLYLQMNSLRAEDTAVYYCARDLMAFNLVRAGGFDVWGQGTLVTVSS

SEQ ID NO: 37 - protein, 107 aa, synthetic construct
  REGION 1..107: note = 78-24D1 VL amino acid sequence - Artificial sequence translatedfrom an isolated clone of an RNA display library
DIQMTQSPSSLSASVGDRVTITCQASQDISNFLNWYQQKPGKAPKLLIYDASNLETGVPSRFSGSGSGTDFTFTISSLQPEDIATYYCQQGDAWPWTFGQGTKVEIK

SEQ ID NO: 38 - protein, 124 aa, synthetic construct
  REGION 1..124: note = 78-24D4 VH amino acid sequence - Artificial sequence translatedfrom an isolated clone of an RNA display library
EVQLVESGGGLVQPGGSLRLSCAASGFTFSSYSMHWVRQAPGKGLEWVSGISFSGRSTSYADSVKGRFTISRDNSKNTLYLQMNSLRAEDTAVYYCARYLMSFNLTRTSWFDVWGQGTLVTVSS

SEQ ID NO: 39 - protein, 107 aa, synthetic construct; identical to SEQ ID NO: 34
  REGION 1..107: note = 78-24D4 VL amino acid sequence - Artificial sequence translatedfrom an isolated clone of an RNA display library

SEQ ID NO: 40 - protein, 124 aa, synthetic construct
  REGION 1..124: note = 78-24D8 VH amino acid sequence - Artificial sequence translatedfrom an isolated clone of an RNA display library
EVQLVKSGGGLVQPGGSLRLSCAASGFTFSSYSMHWVRQAPGKGLEWVSGISGSGRSTSYADSVKGRFTISRDNSKNTLYLQMNSLRAEDTAVYYCARDLMAFNLTRTGGFDVWGQGTLVTVSS

SEQ ID NO: 41 - protein, 107 aa, synthetic construct
  REGION 1..107: note = 78-24D8 VL amino acid sequence - Artificial sequence translatedfrom an isolated clone of an RNA display library
DIQMTQSPSSLSASVGDRVTITCQASQDISNYLNWYQQKPGKAPKLLIYDASNLETGVPSRFSGSGSGTDFTFTINSLQPEDIATYYCQQGDAWPWTFGQGTKVEIK

SEQ ID NO: 42 - protein, 124 aa, synthetic construct
  REGION 1..124: note = 78-24D9 VH amino acid sequence - Artificial sequence translatedfrom an isolated clone of an RNA display library
EVQLVESGGGLVQPGGSLRLSCAASGFTFSSSSMHWVRQAPGKGLEWVSGISGWGGSTSYADSVKGRFTISRDNSKNTLYLQMNSLRAEDTAVYYCARYLMSFNLTRTGWFDVWGQGTLVTVSS

SEQ ID NO: 43 - protein, 107 aa, synthetic construct
  REGION 1..107: note = 78-24D9 VL amino acid sequence - Artificial sequence translatedfrom an isolated clone of an RNA display library
DIQMTQSPSSLSASVGDRVTITCQASQDIANYLNWYQQKPGKAPKLLIYDASNLETGVPSRFSGSGSGTDFTFTISSLQPEDIATYYCQQSDAWPWTFGQGTKVEIK

SEQ ID NO: 44 - protein, 124 aa, synthetic construct
  REGION 1..124: note = 78-24D12 VH amino acid sequence - Artificial sequence translatedfrom an isolated clone of an RNA display library
EVQLVESGGGLVQPGGSLRLSCAASGFTFSSYSMHWVRQAPGKGLEWVSGISGSGLSTSYADSVKGRFTISRDNSKNTLYLQMNSLRAEDTAVYYCARDLMAFNLTRTGGFDVWGQGTLVTVSS

SEQ ID NO: 45 - protein, 107 aa, synthetic construct
  REGION 1..107: note = 78-24D12 VL amino acid sequence - Artificial sequence translatedfrom an isolated clone of an RNA display library
DIQMTQSPSSLSASVGDRVTITCQASHDILNYLNWYQQKPGKAPKLLIYDASNLETGVPSRFSGSGSGTDFTFTISSLQPEDIATYYCQQGHAWLWTFGQGTKVEIK

SEQ ID NO: 46 - protein, 124 aa, synthetic construct
  REGION 1..124: note = 78-24D14 VH amino acid sequence - Artificial sequence translatedfrom an isolated clone of an RNA display library
EVQLVESGGGLVQPGGSLRLSCAASGFTFFSYSMHWVRQAPGKGLEWVSGISGSGWSTSYADSVKGRFTISRDNSKNTLYLQMNSLRAEDTAVYYCVRDLMSFNMTRAGGFDVWGQGTLVTVSS

SEQ ID NO: 47 - protein, 107 aa, synthetic construct; identical to SEQ ID NO: 34
  REGION 1..107: note = 78-24D14 VL amino acid sequence - Artificial sequence translatedfrom an isolated clone of an RNA display library

SEQ ID NO: 48 - protein, 124 aa, synthetic construct
  REGION 1..124: note = 78-24D17 VH amino acid sequence - Artificial sequence translatedfrom an isolated clone of an RNA display library
EVQLVESGGGLVQPGGSLRLSCAASGFTFSSSSMHWVRQAPGKGLEWVSGISGSGGSTSYADSVKGRFTISRDNSKNTLYLQMNSLRAEDTAVYYCARYLMSFNLTRTSWFDVWGQGTLVTVSS

SEQ ID NO: 49 - protein, 124 aa, synthetic construct
  REGION 1..124: note = 78-24D17V VH amino acid sequence - Artificial sequence translatedfrom an isolated clone of an RNA display library
EVQLVESGGGLVQPGGSLRLSCAASGFTFSSSSMHWVRQAPGKGLEWVSGISGSGGSTSYADSVKGRFTISRDNSKNTLYLQMNSLRAEDTAVYYCARYLMSFNLVRTSWFDVWGQGTLVTVSS

SEQ ID NO: 50 - protein, 107 aa, synthetic construct
  REGION 1..107: note = 78-24D17 VL amino acid sequence - Artificial sequence translatedfrom an isolated clone of an RNA display library
DIQMTQSPSSLSASVGDRVTITCQASQAISNYLNWYQQKPGKAPKLLIYDASNLETGVPSRFSGSGSGTDFTFTISSLQPEDIATYYCQQGDAWPWTFGQGTKVEIK

SEQ ID NO: 51 - protein, 124 aa, synthetic construct
  REGION 1..124: note = 78-24D22 VH amino acid sequence - Artificial sequence translatedfrom an isolated clone of an RNA display library
EVQLVESGGGLVQPGGSLRLSCAASGFTFPSYSMHWVRQAPGKGLEWVSGISGSGWSTSYADSVKGRFTISRDNSKNTLYLQMNSLRAEDTAVYYCARDLMAFNLTRAGGFDVWGQGTLVTVSS

SEQ ID NO: 52 - protein, 124 aa, synthetic construct
  REGION 1..124: note = 78-24D22V VH amino acid sequence - Artificial sequence translatedfrom an isolated clone of an RNA display library
EVQLVESGGGLVQPGGSLRLSCAASGFTFPSYSMHWVRQAPGKGLEWVSGISGSGWSTSYADSVKGRFTISRDNSKNTLYLQMNSLRAEDTAVYYCARDLMAFNLVRAGGFDVWGQGTLVTVSS

SEQ ID NO: 53 - protein, 107 aa, synthetic construct; identical to SEQ ID NO: 34
  REGION 1..107: note = 78-24D22 VL amino acid sequence - Artificial sequence translatedfrom an isolated clone of an RNA display library

SEQ ID NO: 54 - protein, 121 aa, synthetic construct
  REGION 1..121: note = 78-28 VH amino acid sequence - Artificial sequence translatedfrom an isolated clone of an RNA display library
EVQLVESGGGLVQPGGSLRLSCAASGFTFSSYYMHWVRQAPGKGLEWVSAIYGNGGYTYYADSVKGRFTISRDNSKNTLYLQMNSLRAEDTAVYYCARGRPARHQVGFDVWGQGTLVTVSS

SEQ ID NO: 55 - protein, 107 aa, synthetic construct
  REGION 1..107: note = 78-28 VL amino acid sequence - Artificial sequence translatedfrom an isolated clone of an RNA display library
DIQMTQSPSSLSASVGDRVTITCQASLDISNYLNWYQQKPGKAPKLLIYDASNLETGVPSRFSGSGSGTDFTFTISSLQPEDIATYYCQQLYTVPLTFGQGTKVEIK

SEQ ID NO: 56 - protein, 125 aa, synthetic construct
  REGION 1..125: note = 78-36 VH amino acid sequence - Artificial sequence translatedfrom an isolated clone of an RNA display library
MEVQLVESGGGLVQPGGSLRLSCVASGFTFSSYYMHWVRQAPGKGLEWVSAISGSGGSTYYADSVKGRFTISRDNSKNTLYLQMNSLRAEDTAVYYCARGRQVGKSSFGEAFDVWGQGTLVTVSS

SEQ ID NO: 57 - protein, 107 aa, synthetic construct
  REGION 1..107: note = 78-36 VL amino acid sequence - Artificial sequence translatedfrom an isolated clone of an RNA display library
DIQMTQSPSSLSASVGDRVTITCQASQDISNYLNWYQQKPGKAPKLLIYDASNLETGVPSRFSGSGSGTDFTFTISSLQPEDIATYYCQQFTSFPYTFGQGTKVEIK

SEQ ID NO: 58 - protein, 123 aa, synthetic construct
  REGION 1..123: note = 78-39 VH amino acid sequence - Artificial sequence translatedfrom an isolated clone of an RNA display library
EVQLVESGGGLVQPGGSLRLSCAASGFTLSSYAMHWVRQAPGKGLEWVSAISWSGGSTNYADSVKGRFTISRDNSKNTLYLQMNSLRAEDTAVYYCARGPRPTYKMSIGFDVWGQGTLVTVSS

SEQ ID NO: 59 - protein, 107 aa, synthetic construct
  REGION 1..107: note = 78-39 VL amino acid sequence - Artificial sequence translatedfrom an isolated clone of an RNA display library
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGQGTKVEIK

SEQ ID NO: 60 - protein, 454 aa, synthetic construct
  REGION 1..454: note = 78-8D2 HC amino acid sequence - Artificial sequence translatedfrom an isolated clone of an RNA display library
EVQLVESGGGLVQPGGSLRLSCAASGFTFSSYVMHWVRQAPGKGLEWVSAITWSGRGTYYADSVKGRFTISRDNSKNTLYLQMNSLRAEDTAVYYCARGRLFGLALYGEAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 454 aa, synthetic construct
  REGION 1..454: note = 78-8D4 HC amino acid sequence - Artificial sequence translatedfrom an isolated clone of an RNA display library
EVQLVESGGGLVQPGGSLRLSCAASGFTFSSYVMHWVRQAPGKGLEWVSAITWSGGGTYYADSVKGRFTISRDNSKNTLYLQMNSLRAEDTAVYYCARGRLFGLVRYGEAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 454 aa, synthetic construct
  REGION 1..454: note = 78-8D22 HC amino acid sequence - Artificial sequence translatedfrom an isolated clone of an RNA display library
EVQLVESGGGLVQPGGSLRLSCAASGFTFSSYVMHWVRQAPGKGLEWVSAISWSGSGTYYADSVKGRFTISRDNSKNTLYLQMNSLRAEDTAVYYCARGRLFGLARYGEAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 214 aa, synthetic construct
  REGION 1..214: note = 78-8 LC amino acid sequence - Artificial sequence translated froman isolated clone of an RNA display library
DIQMTQSPSSLSASVGDRVTITCQASKDISNYLNWYQQKPGKAPKLLIYDASNLETGVPSRFSGSGSGTDFTFTISSLQPEDIATYYCQQFNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 454 aa, synthetic construct
  REGION 1..454: note = 78-24D1V HC amino acid sequence - Artificial sequence translatedfrom an isolated clone of an RNA display library
EVQLVESGGGLVQPGGSLRLSCAASGFTFTSYSMHWVRQAPGKGLEWVSGISGSGRSTSYADSVKGRFTISRDNSKNTLYLQMNSLRAEDTAVYYCARDLMAFNLVRAGGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 454 aa, synthetic construct
  REGION 1..454: note = 78-24D17V HC amino acid sequence - Artificial sequence translatedfrom an isolated clone of an RNA display library
EVQLVESGGGLVQPGGSLRLSCAASGFTFSSSSMHWVRQAPGKGLEWVSGISGSGGSTSYADSVKGRFTISRDNSKNTLYLQMNSLRAEDTAVYYCARYLMSFNLVRTSWFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 454 aa, synthetic construct
  REGION 1..454: note = 78-24D22V HC amino acid sequence - Artificial sequence translatedfrom an isolated clone of an RNA display library
EVQLVESGGGLVQPGGSLRLSCAASGFTFPSYSMHWVRQAPGKGLEWVSGISGSGWSTSYADSVKGRFTISRDNSKNTLYLQMNSLRAEDTAVYYCARDLMAFNLVRAGGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 214 aa, synthetic construct
  REGION 1..214: note = 78-24 LC amino acid sequence - Artificial sequence translatedfrom an isolated clone of an RNA display library
DIQMTQSPSSLSASVGDRVTITCQASQDISNYLNWYQQKPGKAPKLLIYDASNLETGVPSRFSGSGSGTDFTFTISSLQPEDIATYYCQQGDAWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 454 aa, synthetic construct; identical to SEQ ID NO: 64
  REGION 1..454: note = CTLA4-7 HC amino acid sequence - Artificial sequence translatedfrom an isolated clone of an RNA display library

SEQ ID NO: 69 - protein, 214 aa, synthetic construct; identical to SEQ ID NO: 67
  REGION 1..214: note = CTLA4-7 LC amino acid sequence - Artificial sequence translatedfrom an isolated clone of an RNA display library

SEQ ID NO: 70 - protein, 454 aa, synthetic construct; identical to SEQ ID NO: 65
  REGION 1..454: note = CTLA4-8 HC amino acid sequence - Artificial sequence translatedfrom an isolated clone of an RNA display library

SEQ ID NO: 71 - protein, 214 aa, synthetic construct; identical to SEQ ID NO: 67
  REGION 1..214: note = CTLA4-8 LC amino acid sequence - Artificial sequence translatedfrom an isolated clone of an RNA display library

(19) United States
(12) Patent Application Publication
(10) Pub. No.: US 2026/0193353 A1
(43) Pub. Date: Jul. 9, 2026

(54) Anti-CTLA4 monoclonal antibodies and chimeric antigen receptors

(71) Applicant: NantBio, Inc., Culver City, CA (US)

(72) Inventors: Clifford Anders Olson, Culver City, CA (US); Shiho Tanaka, Culver City, CA (US); Kayvan Niazi, Culver City, CA (US); Melanie Hermreck, Culver City, CA (US); Thomas H. King, Culver City, CA (US); Zhimin Guo, Culver City, CA (US)

(73) Assignee: NantBio, Inc., Culver City, CA (US)

(21) Appl. No.: 19/543,619

(22) Filed: Feb. 18, 2026

Related U.S. Application Data
(63) Continuation of application No. 18/001,566, filed on Jun. 9, 2021, now Pat. No. 12,595,307.
(60) Provisional application No. 63/038,111, filed on Jun. 11, 2020.

(51) Int. Cl. C07K 16/28; A61K 49/00; A61P 35/00

(52) U.S. Cl. CPC C07K 16/2818; A61K 49/0002; A61P 35/00; C07K 2317/35; C07K 2317/622

(57) ABSTRACT

Compositions and methods are presented in which selected polypeptide compounds bind to CTLA-4. Most typically, binding is mediated by selected VH and/or VL domains, and preferred compounds are prepared as scFv, IgG, or CAR.

This application is a continuation application of allowed U.S. patent application Ser. No. 18/001,566, which was filed Dec. 12, 2022, and which claims priority to U.S. Provisional Patent Application Ser. No. 63/038,111, which was filed Jun. 11, 2020, and each of which are incorporated by reference in its entirety.

### SEQUENCE LISTING

The content of the XML file of the sequence listing with the file name 102719.0032US.xml, which is 104,407 bytes in size and was created on Jun. 1, 2021 and which was electronically submitted via Patent Center along with the present application, is incorporated by reference in its entirety.

### FIELD OF THE INVENTION

The field of the invention is compositions and methods for compounds that bind to cytotoxic T lymphocyte-associated antigen-4 (CTLA-4), and especially antibodies, antibody fragments, scFvs, and fusion molecules comprising VH and VL domains as presented herein.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Immunotherapy has increasingly become a promising therapeutic strategy against various cancers. Among other options, cancer vaccine compositions, typically in combination with immune stimulatory drugs, have shown in at least some approaches significant therapeutic effect. Unfortunately, the tumor microenvironment often has an immune suppressive effect of T cells and NK cells and immune therapies are often rendered less effective. Cytotoxic T lymphocyte-associated antigen-4 (CTLA-4, CD152) is a membrane glycoprotein expressed by activated effector T cells (Teff) and participates in the repression of T cell proliferation, cell cycle progression and cytokine (IL-2, IFN-γ) production. CTLA-4 is thought to exert its inhibitory function through multiple mechanisms including competition with CD28-positive co-stimulation for binding to their shared B7 ligands (CD80/CD86) on the antigen-presenting cells (APC), as well as direct inhibitory effects through the cytoplasmic tail which associates with signaling molecules.

To overcome immune suppression due to CTLA-4 activity, anti-CTLA-4 antibodies can be used as a therapeutic effector. Currently known anti-CTLA-4 antibodies include ipilimumab and tremelimumab, which have been used, alone or in combination with chemotherapy, cancer vaccines, or other antibodies (anti-PD-1 or anti-OX40), in the treatment of various cancers, including melanoma, non-small cell lung cancer (NSCLC), breast cancer, prostate cancer, pancreatic cancer, hepatocellular carcinoma, and mesothelioma. Ipilimumab produced increased long-term survival at a dose of 10 mg/kg in melanoma and had anti-tumor activity in patients with B-cell lymphoma. However, ipilimumab was not shown effective in patients with metastatic castration-resistant prostate cancer. Although it did not show similar benefit as ipilimumab, clinical trials for tremelimumab demonstrated acceptable tolerability and clinically meaningful activity in patients with melanoma, refractory metastatic colorectal cancer, hepatocellular carcinoma and malignant mesothelioma.

Unfortunately, ipilimumab and tremelimumab have significant adverse effects and are not always effective in reducing immune suppression. Thus, even though various systems and methods of interfering with CTRLA-4 signaling are known in the art, all or almost all of them suffer from several drawbacks. Therefore, there remains a need for compositions and methods for improved compounds that bind to CTLA-4.

### SUMMARY OF THE INVENTION

The inventive subject matter is directed to various compositions and methods of polypeptides that bind with high specificity and affinity to CTLA-4. In preferred embodiments, the polypeptides are configured as scFvs, antibodies, or portions thereof, or as chimeric molecules such as CARs, N-803 or T×M derivatives. In further preferred embodiments, the polypeptides comprise a VH and/or VL domain and have an amino acid sequence as described in SEQ ID NO.1-59, or comprise a heavy or a light chain of an antibody as described in SEQ ID NO: 60-71. As will be readily appreciated, these polypeptides can be employed in various diagnostic and therapeutic uses, and especially in the reduction of immune suppression and treatment of cancer.

In one aspect of the inventive subject matter, the inventors contemplate a single chain variable fragment (scFv) peptide that includes a V_{H} segment comprising a first amino acid sequence selected from the group consisting of SEQ ID NO. 1, 3, 5, 7, 9, 11, 13, 15, 17, 19, 21, 23, 25, 27, 29, 31, 33, 35, 36, 38, 40, 42, 44, 46, 48, 49, 51, 54, 56, and 58, and/or a V_{L} segment comprising a second amino acid sequence selected from the group consisting of SEQ ID NO. 2, 4, 6, 8, 10, 12, 14, 16, 18, 20, 22, 24, 26, 28, 30, 32, 34, 37, 39, 41, 43, 45, 47, 50, 53, 55, 57, and 59. Most typically, the V_{H} segment and V_{L} segment are coupled with a linker peptide (e.g., a glycine-rich peptide). Moreover, it is contemplated that the single chain variable fragment (scFv) peptide may further comprise at least two pairs of the V_{H} segment and V_{L} segment, wherein the at least two pairs are linked to form a multivalent scFv. Where desired the peptide may be present in the pharmacologically acceptable carrier.

In another aspect of the inventive subject matter, the inventors contemplate an antibody that comprises a heavy chain comprising a first amino acid sequence selected from the group consisting of SEQ ID NO. 60, 61, 62, 64, 65, 66, 68, and 70, and/or a light chain comprising a second amino acid sequence selected from the group consisting of SEQ ID NO. 63, 67, 69, and 71. For example, suitable antibodies include those having a heavy chain comprising a first amino acid sequence of SEQ ID NO. 68 and a light chain comprising a second amino acid sequence of SEQ ID NO. 69, or a heavy chain comprising a first amino acid sequence of SEQ ID NO. 70 and a light chain comprising a second amino acid sequence of SEQ ID NO. 71.

In a further aspect of the inventive subject matter, the inventors contemplate a pharmaceutical composition for treating a patient having a cancer that includes a single chain variable fragment (scFv) peptide or an antibody as presented herein, wherein the single chain variable fragment (scFv) peptide or the antibody is present in a pharmacologically acceptable carrier.

In still another aspect of the inventive subject matter, the inventors contemplate a diagnostic composition that comprises a single chain variable fragment (scFv) peptide or an antibody as presented herein wherein the single chain variable fragment (scFv) peptide or the antibody further comprises a detectable label.

Moreover, the inventors also contemplate a recombinant nucleic acid, comprising: first nucleic acid segment encoding a first amino acid sequence selected from the group consisting of SEQ ID NO. 1, 3, 5, 7, 9, 11, 13, 15, 17, 19, 21, 23, 25, 27, 29, 31, 33, 35, 36, 38, 40, 42, 44, 46, 48, 49, 51, 54, 56, and 58, and/or a second amino acid sequence selected from the group consisting of SEQ ID NO. 2, 4, 6, 8, 10, 12, 14, 16, 18, 20, 22, 24, 26, 28, 30, 32, 34, 37, 39, 41, 43, 45, 47, 50, 53, 55, 57, and 59. Preferably, but not necessarily, the first and second segments are present in a same reading frame. Additionally, the inventors contemplate a recombinant nucleic acid that comprises a first nucleic acid segment encoding a first amino acid sequence selected from the group consisting of SEQ ID NO. 60, 61, 62, 64, 65, 66, 68, and 70, and/or a second amino acid sequence selected from the group consisting of SEQ ID NO. 63, 67, 69, and 71. Preferably, but not necessarily, the first and second segments are in a bicistronic arrangement.

For example, contemplated recombinant nucleic acids may comprise a first nucleic acid segment encoding a first amino acid sequence of SEQ ID NO. 68 and second nucleic acid segment encoding a second amino acid sequence of SEQ ID NO. 69, or a first nucleic acid segment encoding a first amino acid sequence of SEQ ID NO. 70 and a second nucleic acid segment encoding a second amino acid sequence of SEQ ID NO. 71.

As will be readily appreciated, the recombinant nucleic acid may further comprise a third segment encoding a polypeptide that encodes at least a portion of a chimeric antigen receptor (CAR), wherein the CAR, when expressed in the cell, has an antigen binding ectodomain that comprises the first and/or second nucleic acid segment. Alternatively, the recombinant nucleic acid may also comprises a third segment encoding a polypeptide that encodes at least a portion of a N-803 or T×M, wherein the N-803 or T×M, when expressed in the cell, has an antigen binding domain that comprises the first and/or second nucleic acid segment.

Therefore, the inventors also contemplate a recombinant cell that includes (e.g., via transfection or genetic engineering) a recombinant nucleic acid as described above. Among other suitable cell types, the recombinant cell may be a recombinant T cell, a recombinant NK cell, a recombinant NKT cell, a recombinant monocyte, a recombinant macrophage, or a recombinant dendritic cell. Alternatively, the recombinant cell may also be a production cell engineered to produce a recombinant protein such as a CHO cell or EC7 cell.

In still further contemplated aspects, a method of reducing an CTLA-4-mediated effect in a cell or tissue is presented that includes a step of contacting the cell or tissue with a single chain variable fragment (scFv) peptide an antibody as presented herein in an amount that reduces the CTALA-4-mediated effect in the cell or tissue.

Consequently, the inventors also contemplate a method of treating a patient having a tumor that includes a step of administering to the patient a pharmaceutical composition that comprises a single chain variable fragment (scFv) peptide or an antibody as presented herein, or a recombinant cell as presented herein. Most preferably, the pharmaceutical composition is administered to the patient in a dose and a schedule effective to treat the tumor.

Therefore, and viewed from a different perspective, the inventors also contemplate a method of reducing immune suppression in a patient having a tumor that includes a step of administering to the patient a pharmaceutical composition that comprises a single chain variable fragment (scFv) peptide or an antibody as presented herein, or a recombinant cell as presented herein. Most preferably, the pharmaceutical composition is administered to the patient in a dose and a schedule effective to reduce immune suppression in a tumor microenvironment.

Thus, use of a single chain variable fragment (scFv) or antibody or recombinant cell as presented herein is contemplated for use in medicine, and especially for treating a cancer and/or reducing immune suppression in a patient having a tumor.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 depicts exemplary SPR results for various scFv constructs as presented herein.

FIGS. 2A-2D depict exemplary results for treated tumors receiving anti-CTLA-4 antibodies and control as presented herein.

FIG. 2E is a Kaplan Meier plot for the treatment groups of FIGS. 2A-2D.

FIGS. 3A-3D depict exemplary results for untreated tumors receiving anti-CTLA-4 antibodies and control as presented herein.

FIG. 3E is a Kaplan Meier plot for the treatment groups of FIGS. 3A-3D.

### DETAILED DESCRIPTION

The inventors have discovered various polypeptides that have significant binding to CTLA-4, and particularly VH and VL domains of antibodies or fragments or fusion proteins containing the VH and/or VL domains (and CDRs contained therein). Most advantageously, the VH and/or VL domains or CDRs contained therein can be used to generate various therapeutic or diagnostic agents with high affinity and specificity towards CTLA-4.

With respect to the VH and VL domains contemplated herein it should be appreciated that the domains generally follow well-known nomenclature. As such, the VH domain denotes the variable fragment of a heavy chain in an antibody that is typically bound to several constant domains (e.g., VH domain bound to CH1, CH2, and CH3 domains in an IgG). Likewise, the VL domain denotes that variable fragment of a light chain in an antibody that is typically bound to a constant domain (e.g., VL domain bound to CL in an IgG). As will therefore be readily appreciated, each of the VH and VL domains will have respective framework regions (FR) and complementarity determining regions (CDR), typically sequentially arranged as FR1-CDR1-FR2-CDR2-FR3CDR3-FR4. Consequently, based on the known framework regions in a VH and VL domain, the skilled artisan will readily recognize the respective CDRs in the VH or VL domain. The terms “domain” and “segment” in their conjunction with VH or VL are used interchangeably herein.

As will be further readily recognized, the skilled artisan will be able to use the amino sequences of VH segments and/or VL segments to generate a recombinant, isolated antibody or fragment thereof. As used herein, the term “antibody” refers to immunoglobulin molecules and immunologically active portions of immunoglobulin molecules, i.e., molecules that contain an antigen-binding site that immune-specifically bind an antigen. Thus, “antibody and the fragment thereof” includes a whole immunoglobulin molecule (e.g., a full-size, whole IgG1, IgA, etc.), and an antigen-binding fragment of the whole immunoglobulin molecule. Thus, contemplated fragments include an scFv, Fab fragments, Fab′ fragments, F(ab′)2, disulfide linked Fvs (sdFvs), Fvs, and any fragment comprising either V_{H} segment and/or V_{L} segment. Where the antibody is an immunoglobulin, it is contemplated that the immunoglobulin can include any type (e.g., IgG, IgE, IgM, IgD, IgA, or IgY) and any class (e.g., IgG₁, IgG₂, IgG₃, IgG₄, IgA₁ or IgA₂) of heavy chain or constant domain to constitute different types of immunoglobulins. In addition, the “antibody” will especially include a human antibody, a humanized antibody, a chimeric antibody, a monoclonal antibody, and a polyclonal antibody. Thus, it should be appreciated that the VH and/or VL domains of SEQ ID NOs. 1-59 can be grafted into any existing (typically human or humanized) antibody or antibody fragment using methods well known in the art.

The inventors further contemplated that the that the amino sequences of VH segments and/or VL segments presented in SEQ ID NOs.1-59 can be used coupled with a carrier protein to generate a hybrid protein having the VH segments and/or VL segments on its surface such that CTLA-4 can be bound or blocked by the hybrid protein. Any suitable form of carrier protein that can stably carry a VH segment and/or a VL segment and preferably provide an access to a tumor microenvironment is contemplated. One especially preferred carrier protein includes albumin, refolded albumin, and other proteins with affinity to antibody portions (e.g., protein A, protein G, protein Z) coupled with one or more VH segments and/or VL segments.

Typically, VH segments and/or VL segments are coupled with an anchor molecule by which VH segments and/or VL segments can be coupled with the carrier protein. For example, where the carrier protein is an albumin, the anchor molecule can be a hydrophobic peptide or glycolipids in any suitable size to fit in one of Sudlow site I and II of the albumin or any other hydrophobic area of the albumin. For example, the recombinant immunoglobulin protein against CTLA-4 as described above can be coupled with the carrier protein via its Fc domain. In other embodiments, the anchor molecule may include a hydrophobic peptide (in a length of at least 10 amino acids, 15 amino acids, 20 amino acids, 30 amino acids, etc.). In these embodiments, various configurations of VH segments and/or VL segments (e.g., as a form of scFv) and hydrophobic peptides can be contemplated. For example, a monovalent scFv domain can be directly linked to a hydrophobic peptide, or a multivalent scFv can be directly linked to a hydrophobic peptide. Alternatively, one scFv domain can be directly linked to a plurality of hydrophobic peptides or a plurality of scFv domain can be directly linked to a plurality of hydrophobic peptides.

Alternatively, or additionally, one or more VH segments and/or VL segments can be coupled with an intermediate molecule that has an anchor portion to bind to the carrier protein. In a preferred embodiment, the inventors contemplate that the intermediate molecule provides a plurality of binding sites for VH segments and/or VL segments such that multiple target recognition domains can be carried via a single binding site on the carrier protein. Suitable intermediate molecule may include any protein, glycolipid, organic molecule, or inorganic molecule that does not provide any significant toxicity to the naïve tissue. For example, the suitable intermediate molecule may include a nanoparticle (e.g., quantum dots, gold nanoparticles, magnetic nanoparticles, nanotubes, polymeric nanoparticles, dendrimers, etc.), or a bead (e.g., polystyrene bead, latex bead, dynabead, etc.). Preferably, the nanoparticle and/or beads have a dimension below 1 μm, preferably below 100 nm. The nanoparticle may be crosslinked to or partially coated with a hydrophobic tail that provide an anchor to the carrier protein (e.g., albumin). One or more VH segments and/or VL segments can be also crosslinked to or partially coated on the nanoparticles (e.g., via an extra tail domain linked to the target recognition domain for crosslinking, etc.).

In another example, suitable intermediate molecules may include beads (e.g., polystyrene beads, latex beads, dynabeads, etc.) coupled with an antibody against the carrier protein. Thus, where the carrier protein is an albumin, the beads can be coupled (e.g., crosslinked, coated, etc.) with α-albumin antibody such that the bead can bind to the carrier protein with a high affinity and specificity. One or more VH segments and/or VL segments can be also crosslinked to or partially coated on the bead (e.g., via an extra tail domain linked to the target recognition domain for crosslinking, thiol-mediated crosslinking, etc.).

In some embodiments, scFv peptide can form a recombinant immunoglobulin protein complex that comprises or mimics an ALT-803 (IL-15 superagonist complex, see e.g., Blood 2015 126:1957) or T×M (targeted ALT-803-based scaffold platform, see e.g., URL altorbioscience.com/our-science/il-15-protein-superagonist-and-scaffold-technology/) structure. Preferably, the inventors contemplate that, where the immunoglobulin protein complex mimics T×M structure, the scFv peptide can be directly (or indirectly via a linker) coupled to one or more interleukin-15 (IL-15) binding motif, and/or one or more ligand to the IL-15 binding motif (e.g., IL-15, IL-15N72D, etc.). Thus, where the recombinant immunoglobulin protein complex mimics T×M IgG1 structure, the recombinant immunoglobulin protein complex may include 1-4 scFv peptide against CTLA-4.

Additionally, the recombinant immunoglobulin protein complex mimicking the T×M structure having one or more scFv peptides coupled with IL-15 binding motif or its ligand may also include a binding domain against a tumor specific antigen or patient- and tumor specific neoepitope (e.g., an scFv peptide against a neoepitope, etc). For example, the recombinant immunoglobulin protein complex may include two scFv peptides against CTLA-4 coupled with two IL-15 binding motives and two ScFv peptides against the neoepitope coupled with two IL-15 binding motif ligands. Preferably, the neoepitope is patient-specific and tumor-specific, which is identified by omics analysis of the sequence data as disclosed, for example, in US 2012/0059670A1 and US 2012/0066001A1.

In some embodiments, the scFv, recombinant antibody or fragment thereof, can be generated using one sequence encoding a VH segment among SEQ ID NO. 1, 3, 5, 7, 9, 11, 13, 15, 17, 19, 21, 23, 25, 27, 29, 31, 33, 35, 36, 38, 40, 42, 44, 46, 48, 49, 51, 54, 56, and 58. In other embodiments, the scFv, recombinant antibody or fragment thereof, can be generated using one sequence encoding a VL segment among SEQ ID NO. 2, 4, 6, 8, 10, 12, 14, 16, 18, 20, 22, 24, 26, 28, 30, 32, 34, 37, 39, 41, 43, 45, 47, 50, 53, 55, 57, and 59. In still other embodiments, the scFv, recombinant antibody or fragment thereof, can be generated using one sequence encoding a VH segment among SEQ ID NO. 1, 3, 5, 7, 9, 11, 13, 15, 17, 19, 21, 23, 25, 27, 29, 31, 33, 35, 36, 38, 40, 42, 44, 46, 48, 49, 51, 54, 56, and 58 and one sequence encoding a VL segment among SEQ ID NO. 2, 4, 6, 8, 10, 12, 14, 16, 18, 20, 22, 24, 26, 28, 30, 32, 34, 37, 39, 41, 43, 45, 47, 50, 53, 55, 57, and 59. In these embodiments, it is preferred that the sequence encoding the VH segment and sequence encoding the VL segment are paired having a common designator in the sequence listing with a distinguishing VH and VL identifier (e.g., 73-2 VH and 73-2 VL as shown in SEQ ID NO. 1 and 2, respectively). However, as is shown in more detail below, it is also contemplated that any pair of VH segment and VL segment can be generated to form a CTLA-4 binding molecule by selecting one VH segment from SEQ ID No. xx and one VL segment from SEQ ID No. yy, with xx being any one of 1, 3, 5, 7, 9, 11, 13, 15, 17, 19, 21, 23, 25, 27, 29, 31, 33, 35, 36, 38, 40, 42, 44, 46, 48, 49, 51, 54, 56, and 58, and with yy being any one of 2, 4, 6, 8, 10, 12, 14, 16, 18, 20, 22, 24, 26, 28, 30, 32, 34, 37, 39, 41, 43, 45, 47, 50, 53, 55, 57, and 59.

The inventors also contemplate that the scFv, recombinant antibody or fragment thereof, can be generated with amino acid sequences encoding VH segment and/or VL segment that are at least 85% identical, preferably at least 90% identical, more preferably at least 95% identical to any one of SEQ ID No. xx or yy. Most typically, where the identity is not 100%, the differing amino acid(s) will be located in one or more of the framework regions of the sequences and will not affect the CDR regions of a given sequence. In further embodiments, it is preferred that the binding affinity of the scFv peptide, recombinant antibody or fragment thereof, is no higher than 100 nM, or no higher than 50 nM, or no higher than 20 nM, or no higher than 10 nM, or no higher than 5 nM, or no higher than 2 nM, or no higher than 1 nM. For example, one could select any scFv with a specific VH and/or VL domain as presented herein and then subjected the scFv to affinity maturation via random mutagenesis in the CDR regions for VH and VL. In such examples, it is typically contemplated that no more than 4, or nor more than 3, or no more than 2 amino acids will be changed across all CDR regions of a given sequence. As will be recognized, multiple binders can therefore be isolated from the affinity maturation process with improved binding characteristics.

Most typically, the VH segment and VL segment in an scFv peptide or similar construct are coupled via a linker or a spacer, which is typically between 5-40 amino acids, preferably between 10-30 amino acids, more preferably between 20-30 amino acids. In some embodiments, the linker can couple the N-terminus of VH segment and C-terminus of VL segment. In other embodiments, the linker can couple the N-terminus of VL segment and C-terminus of VH segment. The inventors further contemplate that glycine-rich sequences (e.g., (G4S) n, with n between 1-5, etc.) for the linker are preferred to provide structural flexibility between the VH segment and VL segments. It is also contemplated that the linker includes one or more serine or threonine residue to increase solubility of the scFv peptide, recombinant antibody or fragment thereof. There are numerous linkers and methods of making scFv known in the art, and all such known methods are deemed suitable for use herein.

Additionally, the scFv peptide can include a plurality of VH segments and VL segments to form a divalent or multivalent scFv. In some embodiments, the plurality of VH segments and/or VL segments may have same amino sequences (e.g., a multivalent scFv having three VH segments and three VL segments, and all VH segments have the same VH and all VL segments have the same VL domain. In other embodiments, at least two of VH segments and/or VL segments may have different amino acid sequences (e.g., a multivalent scFv having three VH segments and three VL segments, and two of VH segments are identical VH domains and one of VH segments is distinct, and two of VL segments are identical VL domains and one of VH segments is distinct.

Preferably, the binding affinity (Kd) of the scFv, recombinant antibody or fragment thereof, to CTLA-4 (at least one of 72-mer and 77-mer IL-8) is at least less than 1×10⁻⁷M, preferably less than 1×10⁻⁸M, and more preferably less than 1×10⁻⁹M, measured at a temperature between 25° C. to 37° C., and measured in a pH range between 5.5-7.5. The inventors contemplate that the binding affinities of the scFv, recombinant antibody or fragment thereof, to CTLA-4 may be different due to the structural differences even if the scFv, recombinant antibody or fragment thereof, are generated using the same amino acid sequences of V_{H} segment and/or V_{L} segments. For example, Tables 1-4 provide different affinities (measured in KD value) of different scFv clones, while Table 5 shows different affinities (measured in KD value) of different recombinant antibody (IgG1) clones.

It is further contemplated that the scFv peptide (or VH segment and VL segment of an antibody or fragment thereof) can be encoded by a single recombinant nucleic acid. In such embodiment, the recombinant nucleic acid includes at least two nucleic acid segments: a first nucleic acid segment encoding the VH segment and a second nucleic acid segment encoding the VL segment of the inventive subject matter. However, it is also contemplated that the first and second nucleic acid segments that the encode VH and VL segment respectively that are at least 85% identical, preferably at least 90% identical, more preferably at least 95% identical to any one of SEQ ID No. 1-59. Most preferably, the two nucleic acid segments are in the same reading frame such that two nucleic acid segments can be translated into a single protein having two peptide segments.

Additionally, the recombinant nucleic acid may include a third nucleic acid segment between the first and second nucleic acid segment encoding a linker peptide (preferably G-rich or otherwise flexible linker peptide), which is typically between 5-40 amino acids, preferably between 10-30 amino acids, more preferably between 20-30 amino acids. In this embodiment, it is especially preferred that the three nucleic acid segments are in the same reading frame such that three nucleic acid segments can be translated into a single protein having three peptide segments.

On the other hand, where the CTLA-4 binding molecule is an antibody (e.g., IgG), the VH segment and VL segment as presented herein may be encoded on single recombinant bicistronic construct or the segments may be encoded on two separate constructs. Most typically, the nucleic acid encoding the VH domain will also encode in frame one or more CH domains (e.g., CH1-CH2-CH3 in case of an IgG). Likewise, the nucleic acid encoding the VL domain will also encode in frame one CH domain (e.g., CL in case of an IgG). Most typically, the constant domains for heavy and light chains will be human humanized, but other mammalian sequences are also deemed appropriate. Moreover, it should be recognized that the recombinant nucleic acids will preferably (but not necessarily) have a codon usage that is optimized for the cell expressing the antibody.

The inventors further contemplate that the scFv peptide, antibodies or fragments thereof, can be formulated as a pharmaceutical composition so that it can be administered to the patient having a tumor to reduce or inhibit signaling and attendant immune suppression by CTLA-4. Therefore, it is contemplated that the scFv peptide, antibodies or fragments thereof, can be formulated in any pharmaceutically acceptable carrier (e.g., as a sterile injectable composition) in an amount of at least 1 ml, preferably at least 5 ml, more preferably and at least 20 ml per dosage unit for a therapeutic formulation. However, alternative formulations are also deemed suitable for use herein, and all known routes and modes of administration are contemplated herein. As used herein, the term “administering” refers to both direct and indirect administration of the compounds and compositions contemplated herein, where direct administration is typically performed by a health care professional (e.g., physician, nurse, etc.), while indirect administration typically includes a step of providing or making the compounds and compositions available to the health care professional for direct administration. In some embodiments, the pharmaceutical formulation is administered via systemic injection including subcutaneous, subdermal injection, or intravenous injection. In other embodiments, and as shown in more detail below, it is contemplated that the formulation is administered via intratumoral injection.

One exemplary method and use of pharmaceutical composition including scFv peptide, the antibody or fragment thereof, which includes VH and VL segments described above is to reduce CTLA-4 mediated effects in a target tissue or target cell. As used herein, the CTLA-4 mediated effect refers to any biological consequence induced directly or indirectly by activity of CTLA-4 in the tissue or the microenvironment of the tissue. Most typically, the effect is observable as a reduction in T cell activity (and especially cytotoxic cell killing by T cells and NK cells), which may be enhanced or observed in a tumor microenvironment. Viewed from a different perspective, the reduction of IL-8 mediated effects in vivo can also be observed by physiological phenomena. For example, reduced CTLA-4 signaling can be observed via reduction or abolishment of tumor growth, by tumor shrinkage, tumor cell apoptosis or necrosis. Similarly, reduced CTLA-4 signaling in a patient and especially in a tumor microenvironment will reduce the tendency of a tumor to produce local or distant metastases.

With respect to dose and schedule of the pharmaceutical composition administration to a patient, it is contemplated that the dose and/or schedule may vary depending on the type of peptides (e.g., scFv, an antibody, an antibody fragment, combination of any two of those, combination of all, etc.), type and prognosis of disease (e.g., tumor type, size, location), health status of the patient (e.g., including age, gender, etc.). While it may vary, the dose and schedule may be selected and regulated so that the formulation does not provide any significant toxic effect to the host normal cells, yet sufficient to be reduce CTLA-4 effect in the tumor microenvironment at least 20%, preferably at least 30%, more preferably at least 40%, most preferably at least 50% within less than 3 hours, 6 hours, 12 hours, 24 hours, 72 hours, or a week. For example, where the CTLA-4 binder is an antibody, suitable doses of the antibody may be between 0.1-1.0 mg, or between 1.0-5.0 mg, or between 5.0-10.0 mg, or between 10-50 mg, or between 50-100 mg per administration.

Additionally, the inventors contemplate that the effect of scFv, antibody or fragment thereof against CTLA-4 can be boosted by co-administration of one or more cancer medications. The cancer medications includes, but not limited to, Fulvestrant, Aldoxorubicin, Docetaxel, a tumor necrosis treatment agent (e.g., ¹³¹I-chTNT-3, etc.), Avelumab (a human monoclonal IgG₁ antibody that blocks interaction between PD-L1 and its receptor), Brachyury-targeting vaccine (e.g., ETBX-051 (Ad5 [E1-, E2b-]-Brachyury)), Her2-targeting vaccine (e.g., ETBX-021, etc.), MUC-1-targeting vaccine (e.g., ETBX-061 (Ad5 [E1-, E2b-]-MUC1)), and yeast vaccines (e.g., GI-4000 (GI-4014, GI-4015, GI-4016, GI-4020), GI-6207, GI-6301), and adenovirus or yeast based vaccines against tumor specific neoepitopes. Exemplary details of these cancer medications are described in PCT/US17/40297, which is incorporated herein as a reference in its entirety.

Where desired, the CTLA-4 binding agent may also be co-administered with a cell-based treatment (at the same time or separated by one or more days), and especially preferred cell-based treatments include transfusion with NK cells and/or T cells. Of course, it should be appreciated that such cells may be genetically modified to express one or more recombinant molecules to so increase target specific tumor cell killing.

In especially preferred aspects, such modified NK cells or T cells may be engineered to express a chimeric antigen receptor (CAR). In some embodiments, the CAR of the NK cells or T cells may target one or more cancer- or tumor specific antigens, while in especially contemplated embodiments the CAR expressed by the NK cells or T cells will target CTLA-4. In general, it is noted that suitable CARs comprise a single-chain variable fragment (scFv) linked to at least one intracellular signaling domain. The scFv recognizes and binds an antigen on the target cell (e.g., antigen or CTLA-4) and triggers effector cell activation. The signaling domains contain in most cases immunoreceptor tyrosine-based activation domains (ITAMs) that are important for intracellular signaling by the receptor. Suitable CARs include first, second, and third generation CARs that can be expressed in a variety of cells, and especially T cells and NK cells.

First generation CARs will typically contain one cytoplasmic signaling domain. For example, one version of a first-generation CAR in T-cells may include a signaling domain from the Fc epsilon receptor gamma (FcεRIγ) that contains one ITAM, while another version may contain the signaling domain from CD3ζwhich contains three ITAMs. Second generation CARs typically employ CD28 as the most common intracellular costimulatory domain alongside CD3ζ. Alternatively, second generation CARs may also incorporate the 4-1BB intracellular signaling domain along with CD3ζ. Third generation CARs will typically include CD3ζ, CD28, and 4-1BB intracellular signaling domains. See also WO 2016/201304 and WO 2018/076391 for further third generation CARs.

### Examples

Affinity of selected scFv molecules to human CTLA-4 was performed via surface plasmon resonance (SPR). More specifically, a Pioneer FE instrument was used to measure the binding affinities between scFv and the recombinant CTLA-4 protein at 25° C. Briefly, biotinylated anti-FLAG M2 antibody was immobilized onto the PCH biosensor (Molecular Devices/ForteBio) coated with neutravidin. 3×FLAG-tagged scFvs were captured with anti-FLAG M2 on the sensor and the binding affinities were measured using OneStep injection with recombinant human CTLA-4 protein. Exemplary results for selected scFvs are shown in Tables 1 and 2 below. In the clone designations of the tables 1-4 below, it should be noted that the clone for the scFv includes both, the VH and VL domain with the SEQ ID NOs as indicated.

TABLE 1
Affinity for scFvs to human CTLA-4

| Clone | K_{D} (nM) |
|---|---|
| 73-2 (SEQ ID NO: 1, SEQ ID NO: 2) | 0.56 |
| 74-1 (SEQ ID NO: 3, SEQ ID NO: 4) | 1.92 |
| 78-8 (SEQ ID NO: 11, SEQ ID NO: 12) | 168 |
| 78-8D2 (SEQ ID NO: 13, SEQ ID NO: 14) | 0.37 |
| 78-8D4 (SEQ ID NO: 15, SEQ ID NO: 16) | 0.21 |
| 78-8D5 (SEQ ID NO: 17, SEQ ID NO: 18) | 0.74 |
| 78-8D6 (SEQ ID NO: 19, SEQ ID NO: 20) | 0.24 |
| 78-8D7 (SEQ ID NO: 21, SEQ ID NO: 22) | 0.30 |
| 78-8D10 (SEQ ID NO: 23, SEQ ID NO: 24) | 0.58 |
| 78-8D14 (SEQ ID NO: 25, SEQ ID NO: 26) | 0.32 |
| 78-8D22 (SEQ ID NO: 27, SEQ ID NO: 28) | 0.16 |

TABLE 2
Affinity for scFvs to human CTLA-4

| Clone | K_{D} (nM) |
|---|---|
| 78-24 (SEQ ID NO: 33, SEQ ID NO: 34) | 136 |
| 78-24D1 (SEQ ID NO: 35, SEQ ID NO: 36) | 1.15 |
| 78-24D4 (SEQ ID NO: 38, SEQ ID NO: 39) | 1.17 |
| 78-24D8 (SEQ ID NO: 40, SEQ ID NO: 41) | 1.46 |
| 78-24D9 (SEQ ID NO: 42, SEQ ID NO: 43) | 1.18 |
| 78-24D12 (SEQ ID NO: 44, SEQ ID NO: 45) | 2.88 |
| 78-24D14 (SEQ ID NO: 46, SEQ ID NO: 47) | 4.6 |
| 78-24D17 (SEQ ID NO: 48, SEQ ID NO: 50) | 0.87 |
| 78-24D22 (SEQ ID NO: 51, SEQ ID NO: 53) | 0.85 |

Similarly, affinity of selected scFv molecules to murine CTLA-4 was performed via surface plasmon resonance (SPR). More specifically, a Pioneer FE instrument was used to measure the binding affinities between scFv and the recombinant CTLA-4 protein at 25° C. Briefly, biotinylated anti-FLAG M2 antibody was immobilized onto the PCH biosensor (Molecular Devices/ForteBio) coated with neutravidin. 3×FLAG-tagged scFvs were captured with anti-FLAG M2 on the sensor and the binding affinities were measured using OneStep injection with recombinant mouse CTLA-4 protein. Exemplary results for selected scFvs are shown in Tables 3 and 4 below.

TABLE 3
Affinity for scFvs to mouse CTLA-4

| Clone | scFv K_{D} (nM) |
|---|---|
| 78-1 (SEQ ID NO: 5, SEQ ID NO: 6) | 0.61 |
| 78-2 (SEQ ID NO: 7, SEQ ID NO: 8) | 0.55 |
| 78-5 (SEQ ID NO: 9, SEQ ID NO: 10) | 0.64 |
| 78-8 (SEQ ID NO: 11, SEQ ID NO: 12) | 0.06 |
| 78-8D2 (SEQ ID NO: 13, SEQ ID NO: 14) | 0.30 |
| 78-8D4 (SEQ ID NO: 15, SEQ ID NO: 16) | 0.20 |
| 78-8D5 (SEQ ID NO: 17, SEQ ID NO: 18) | 0.42 |
| 78-8D6 (SEQ ID NO: 19, SEQ ID NO: 20) | 0.19 |
| 78-8D7 (SEQ ID NO: 21, SEQ ID NO: 22) | 0.39 |
| 78-8D10 (SEQ ID NO: 23, SEQ ID NO: 24) | 0.49 |
| 78-8D14 (SEQ ID NO: 25, SEQ ID NO: 26) | 0.17 |
| 78-8D22 (SEQ ID NO: 27, SEQ ID NO: 28) | 0.37 |
| 78-13 (SEQ ID NO: 29, SEQ ID NO: 30) | 0.79 |

TABLE 4
Affinity for scFvs to mouse CTLA-4

| Clone | scFv K_{D} (nM) |
|---|---|
| 78-19 (SEQ ID NO: 31, SEQ ID NO: 32) | 2.39 |
| 78-24 (SEQ ID NO: 33, SEQ ID NO: 34) | 2.17 |
| 78-24D1 (SEQ ID NO: 35, SEQ ID NO: 37) | 0.059 |
| 78-24D4 (SEQ ID NO: 38, SEQ ID NO: 39) | 1.03 |
| 78-24D8 (SEQ ID NO: 40, SEQ ID NO: 41) | 0.01 |
| 78-24D9 (SEQ ID NO: 42, SEQ ID NO: 43) | 1.11 |
| 78-24D12 (SEQ ID NO: 44, SEQ ID NO: 45) | 0.01 |
| 78-24D14 (SEQ ID NO: 46, SEQ ID NO: 47) | 0.19 |
| 78-24D17(SEQ ID NO: 48, SEQ ID NO: 50) | 1.06 |
| 78-24D22 (SEQ ID NO: 51, SEQ ID NO: 53) | 0.05 |
| 78-28 (SEQ ID NO: 54, SEQ ID NO: 55) | 2.57 |
| 78-36 (SEQ ID NO: 56, SEQ ID NO: 57) | 0.98 |
| 78-39 (SEQ ID NO: 58, SEQ ID NO: 59) | 0.97 |

In still further experiments, selected VH and VL domains were grafted into a human IgG scaffold, and K_{D} determination was performed by SPR (Pioneer FE) or Octet (Red96e) at 25° C. or 37° C. All values shown in Table 5 are x 10⁻⁹ M. Antibodies were captured on the chip surface using anti-human Fc antibody (SPR) or AHC sensor (Octet). Human or mouse CTLA-4 was the analyte as indicated in Table 5.

TABLE 5
KD (nM) for IgG antibodies to human or mouse CTLA-4

| | | 25 deg C. | | 37 deg C. | |
|---|---|---|---|---|---|
| VH | VL | hCTLA-4 | mCTLA-4 | hCTLA-4 | mCTLA-4 |
| 78-8D2 HC (SEQ ID NO: 60) | 78-8 LC (SEQ ID NO: 63) | 0.41 | 0.32 | 0.67 | 0.40 |
| 78-8D4 HC (SEQ ID NO: 61) | 78-8 LC (SEQ ID NO: 63) | 0.39 | 0.27 | 0.39 | 0.27 |
| 78-8D22 HC (SEQ ID NO: 62) | 78-8 LC (SEQ ID NO: 63) | 0.46 | 0.39 | 0.44 | 0.53 |
| 78-24D1V HC (SEQ ID NO: 64) | 78-24 LC (SEQ ID NO: 67) | 1.6 | 0.18 | 1.5 | 0.23 |
| 78-24D17V HC (SEQ ID NO: 65) | 78-24 LC (SEQ ID NO: 67) | 0.84 | 0.34 | 0.67 | 0.33 |
| 78-24D22V HC (SEQ ID NO: 66) | 78-24 LC (SEQ ID NO: 67) | 1.4 | 0.19 | 1.3 | 0.27 |

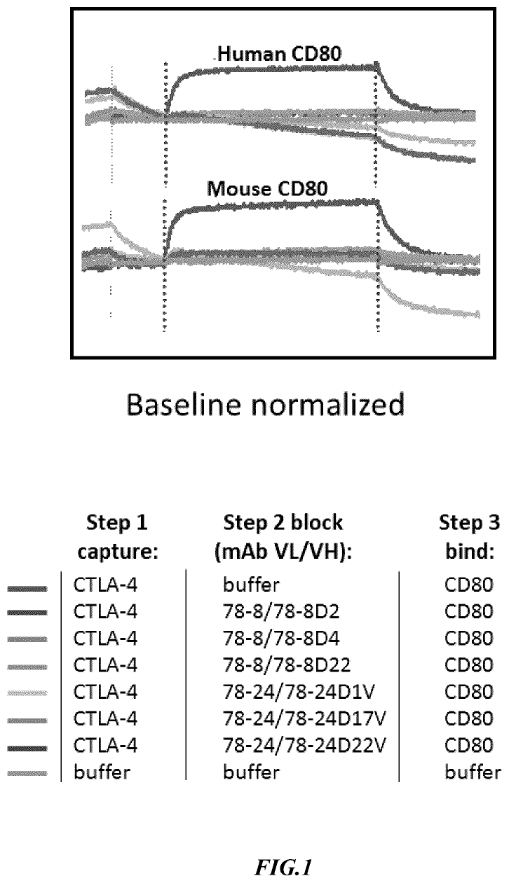

The inventors further investigated antibody (IgG1) blocking CD80/CTLA4 complex by BLI using Human CTLA-4/human CD80 or mouse CTLA-4/mouse CD80. Here, an Octet Red96e instrument was used to test the ability of αCTLA-4 antibodies to block CD80 binding to CTLA-4. Briefly, biotinylated CTLA-4 was immobilized onto SA biosensors (Molecular Devices/ForteBio). 10 μg/mL (67 nM) αCTLA-4s were then used to load the CTLA-4 coated biosensor. Finally, 250 nM CD80 was used to test binding to CTLA-4 in the presence of αCTLA-4 antibodies, and exemplary results are shown in FIG. 1.

Following the above in vitro studies, the inventors then set out to perform various in vivo experiments to investigate the antitumoral activity of selected IgG constructs. Specifically, the inventors tested in a B16F10 mouse tumor model intratumoral immunization using yeast lysate (pTK170, yeast expressing tumor specific neoepitopes), an anti-OX40 antibody, and selected anti-CTLA-4 antibodies as shown. Four groups of animals were used: 1. PBS; 2. pTK170+aOX40+a-CTLA4-7 (SEQ ID NO:68, SEQ ID NO:69); 3. pTK170+aOX40+a-CTLA4-8 (SEQ ID NO:70, SEQ ID NO:71); and 4. pTK170+aOX40+a-CTLA4-9H10. The administered dose of pTK170 yeast was 5 YU/injection, and anti-OX40 and anti-CTLA4 were administered at 100 μg each per injection.

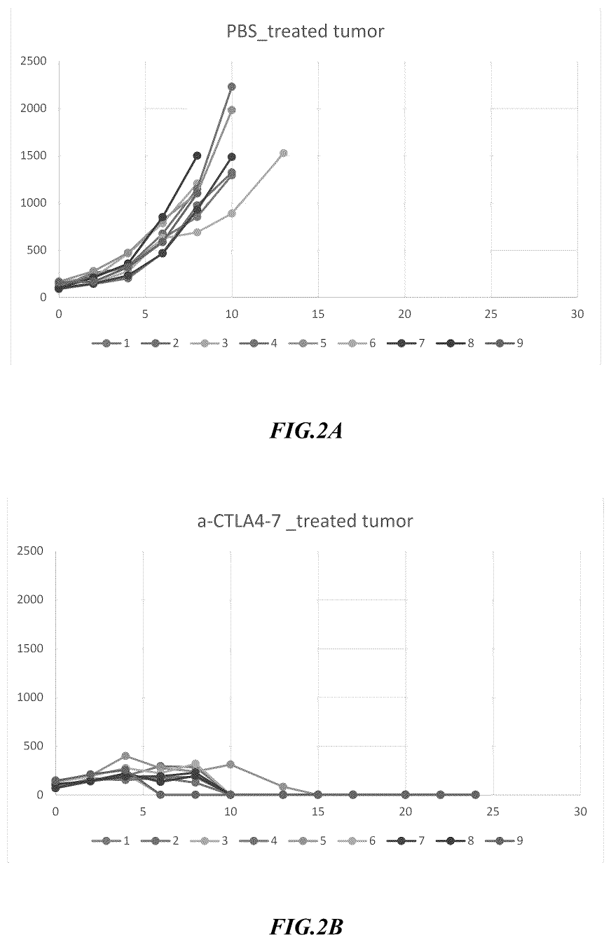

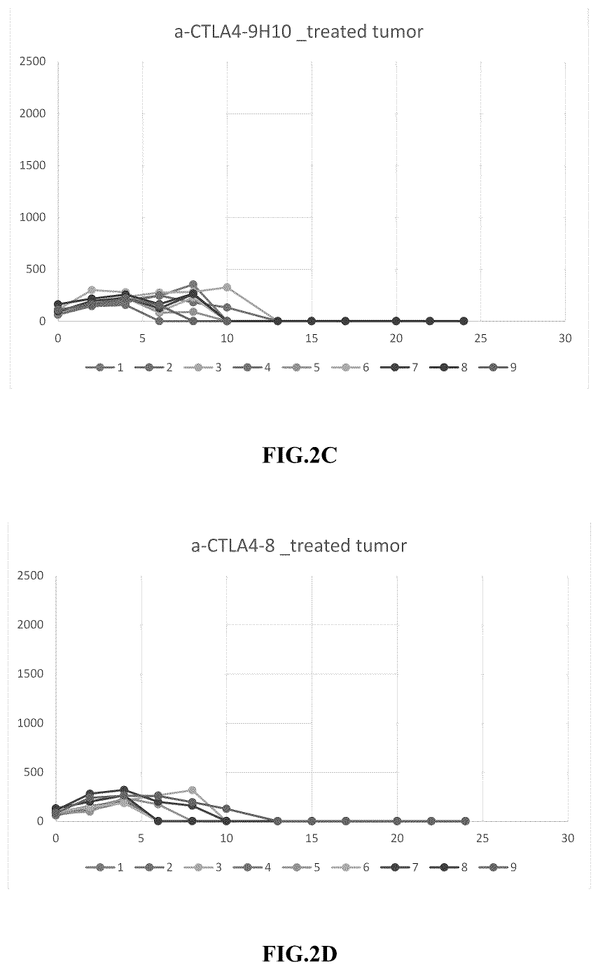

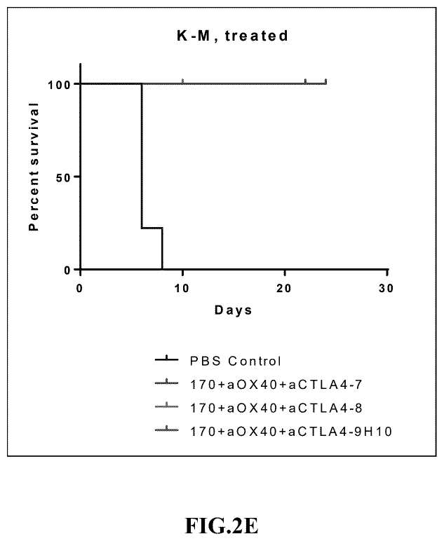

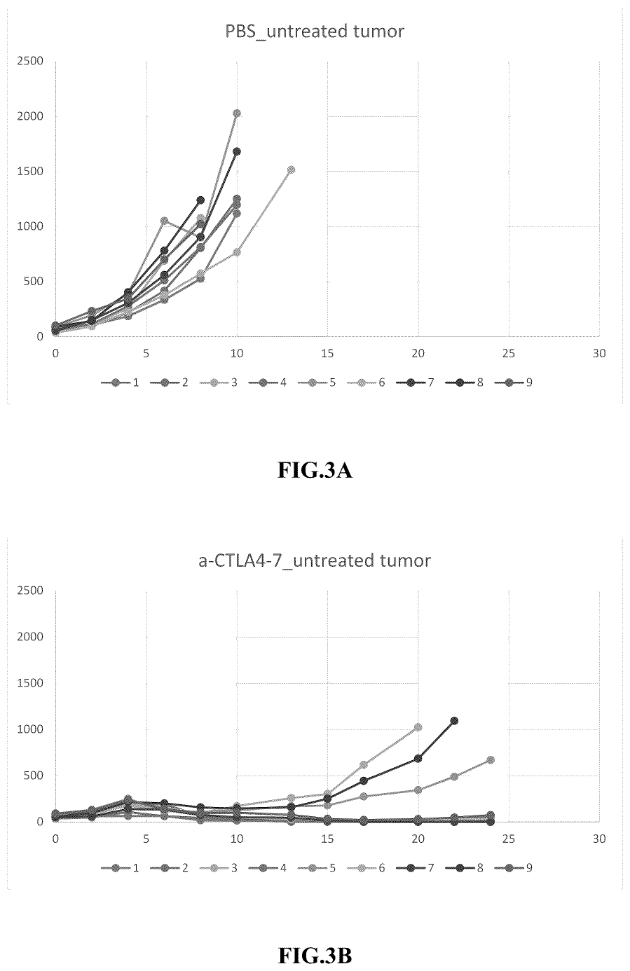

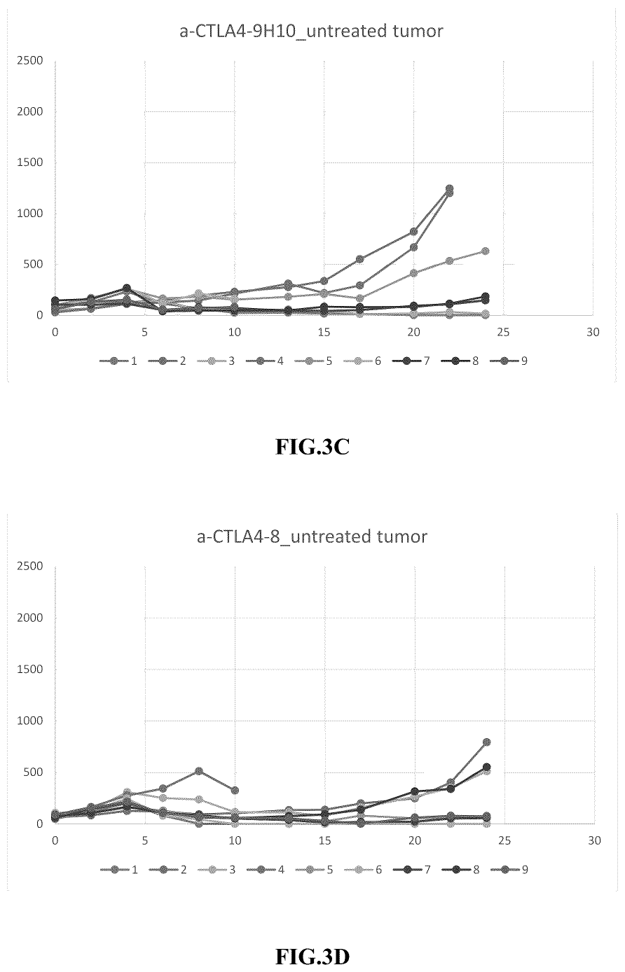

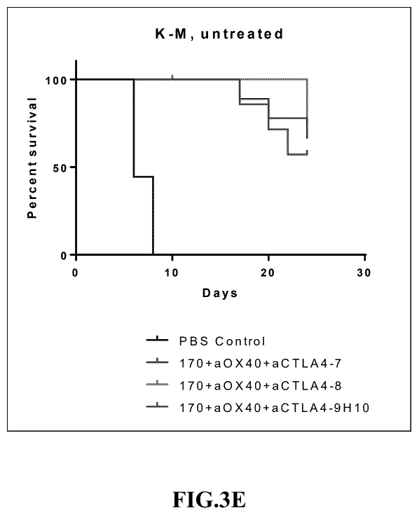

Exemplary results for tumor volumes are shown in FIGS. 2A-2D, and a Kaplan-Meyer Plot for the animals is provided in FIG. 2E. As can be readily seen, all anti-CTLA4 groups (FIGS. 2B-2D) significantly reduce the tumor growth for both treated and untreated tumors compared to PBS (FIG. 2A). There are no statistically significant differences as yet between clone 7, 8 and 9H10 at the 100 μg dose. Non-injected tumor volumes are shown in FIGS. 3A-3D, with FIG. 3A being the PBS control, and FIGS. 3B-3D showing treatment with anti-CTLA4 compounds. The corresponding Kaplan-Meyer Plot for the animals is provided in FIG. 3E.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of treating a patient having a tumor, the method comprising:
administering to the patient a pharmaceutical composition comprising an antibody or fusion protein comprised thereof, the antibody comprising
a variable heavy chain (VH) segment and a variable light chain (VL) segment respectively consisting of SEQ ID NO:60 and SEQ ID NO:63, SEQ ID NO: 61 and SEQ ID NO:63, SEQ ID NO:62 and SEQ ID NO:63, SEQ ID NO: 64 and SEQ ID NO:67, SEQ ID NO:65 and SEQ ID NO:67, SEQ ID NO: 66 and SEQ ID NO:67, SEQ ID NO:68 and SEQ ID NO:69, or SEQ ID NO: 70 and SEQ ID NO:71.

2. The method of claim 1, wherein the antibody comprises a heavy chain comprising a first amino acid sequence of SEQ ID NO. 68 and a light chain comprising a second amino acid sequence of SEQ ID NO. 69, or a heavy chain comprising a first amino acid sequence of SEQ ID NO. 70 and a light chain comprising a second amino acid sequence of SEQ ID NO. 71.

3. The method of claim 1, wherein the pharmaceutical composition is administered to the patient in a dose and a schedule effective to treat the tumor.

4. The method of claim 3, wherein the antibody is administered at a dose of between 0.1-1.0 mg, between 1.0-5.0 mg, between 5.0-10.0 mg, between 10-50 mg, or between 50-100 mg.

5. The method of claim 1, wherein the pharmaceutical composition further comprises a cancer medication.

6. The method of claim 5, wherein the cancer medication comprises Fulvestrant, Doxorubicin, a taxane, a tumor necrosis treatment agent, a PD-L1 inhibiting antibody, or an adenoviral or a yeast-based vaccine against a tumor specific antigen or neoepitope.

7. The method of claim 6, wherein the adenoviral or yeast-based vaccine comprises a Brachyury-targeting vaccine, a Her2-targeting vaccine, a MUC-1-targeting vaccine, or a neoepitope-targeting vaccine.

8. The method of claim 1, wherein the pharmaceutical composition further comprises a cell-based treatment.

9. The method of claim 8, wherein the cell-based treatment comprises a natural killer (NK) or a T cell.

10. The method of claim 9, wherein the NK or T cell is modified to express a chimeric antigen receptor (CAR), wherein the CAR targets a checkpoint inhibitor or a tumor-specific antigen.

11. A method of reducing immune suppression in a patient having a tumor, the method comprising:
administering to the patient a pharmaceutical composition comprising an antibody or fusion protein comprised thereof, the antibody comprising
a variable heavy chain (VH) segment and a variable light chain (VL) segment respectively consisting of SEQ ID NO:60 and SEQ ID NO:63, SEQ ID NO: 61 and SEQ ID NO:63, SEQ ID NO:62 and SEQ ID NO:63, SEQ ID NO: 64 and SEQ ID NO:67, SEQ ID NO:65 and SEQ ID NO:67, SEQ ID NO: 66 and SEQ ID NO:67, SEQ ID NO:68 and SEQ ID NO:69, or SEQ ID NO: 70 and SEQ ID NO:71.

12. The method of claim 11, wherein the antibody comprises a heavy chain comprising a first amino acid sequence of SEQ ID NO. 68 and a light chain comprising a second amino acid sequence of SEQ ID NO. 69, or a heavy chain comprising a first amino acid sequence of SEQ ID NO. 70 and a light chain comprising a second amino acid sequence of SEQ ID NO. 71.

13. The method of claim 11, wherein the pharmaceutical composition is administered to the patient in a dose and a schedule effective to reduce immune suppression.

14. The method of claim 13, wherein the antibody is administered at a dose of between 0.1-1.0 mg, between 1.0-5.0 mg, between 5.0-10.0 mg, between 10-50 mg, or between 50-100 mg.

15. The method of claim 11, wherein the pharmaceutical composition further comprises a cancer medication.

16. The method of claim 15, wherein the cancer medication comprises Fulvestrant, Doxorubicin, a taxane, a tumor necrosis treatment agent, a PD-L1 inhibiting antibody, or an adenoviral or a yeast-based vaccine against a tumor specific antigen or neoepitope.

17. The method of claim 16, wherein the adenoviral or yeast-based vaccine comprises a Brachyury-targeting vaccine, a Her2-targeting vaccine, a MUC-1-targeting vaccine, or a neoepitope-targeting vaccine.

18. The method of claim 11, wherein the pharmaceutical composition further comprises a cell-based treatment.

19. The method of claim 18, wherein the cell-based treatment comprises a natural killer (NK) or a T cell.

20. The method of claim 19, wherein the NK or T cell is modified to express a chimeric antigen receptor (CAR), wherein the CAR targets a checkpoint inhibitor or a tumor-specific antigen.
